(19) United States
(12) Patent Application Publication
(10) Pub. No.: US 2015/0010600 A1
(43) Pub. Date: Jan. 8, 2015

(54) GELLED COSMETIC COMPOSITIONS WITH ENCAPSULATED FRAGRANCE

(71) Applicant: Conopco Inc., d/b/a UNILEVER, Englewood Cliffs, NJ (US)

(72) Inventors: Sarah Jayne Clare, Bebington (GB); Martin Peter Cropper, Birkenhead (GB); Kevin Ronald Franklin, Meols (GB); Anthony Hackett, Warrington (GB); Craig Warren Jones, Port Sunlight (GB); Adam John Limer, Newton-Le-Willows (GB)

(73) Assignee: CONOPCO INC., D/B/A UNILEVER, Englewood Cliffs, NJ (US)

(21) Appl. No.: 14/370,864

(22) PCT Filed: Dec. 18, 2012
(86) PCT No.: PCT/EP2012/075894

§ 371 Date: Jul. 7, 2014

(30) Foreign Application Priority Data
Jan. 18, 2012 (EP) 12151618.1

(51) Int. Cl. A61K 8/11; A61K 8/04; A61Q 15/00; A61K 8/02

(52) U.S. Cl. CPC A61K 8/11; A61K 8/0229; A61K 8/042; A61Q 15/00; A61K 2800/56
USPC 424/401

(57) ABSTRACT

Disclosed are gel compositions that include (a) polyhydric alcohol, (b) water, (c) gelling agent, (d) fragrance-delivery particles having i) at least one shell formed by a step-growth polymerization reaction, ii) interior said shell, a core comprising at least one region formed by chain-grown polymerization of selected monomers, iii) optionally, surfactant grafted to said shell, wherein the fragrance delivery particles have an average diameter of less than 20 microns, and (e) free fragrance, wherein the compositions are in the form of deodorant gel sticks.

### BACKGROUND OF THE INVENTION

This invention relates to cosmetic compositions in the form of gelled sticks, alternatively referred to as “gel sticks” and, more particularly, to gelled cosmetic compositions in the form of deodorant sticks for application to the human axillae, especially the underarms, to reduce malodour, which compositions provide fragrance release over time.

As used herein, “gel sticks” refer to products wherein the gelling agent, together with water and other solvents present therein, form a gel network structure. Such products are commonly translucent or transparent in appearance as distinguished from sticks having one or more waxy materials as the primary structurant thereof, which sticks are commonly opaque.

To provide an extended fragrance benefit in deodorant compositions, formulators may include one or more encapsulates, also known as encaps, that release fragrance in response to a particular stimulus, for example, moisture or shear. Fragrance encapsulates may be used in place of or together with free fragrance which may be the same or different from that used in the encapsulates.

Fragrance encapsulates made from starch, cyclodextrin or gelatin are among the encaps commonly used in anhydrous cosmetic compositions, including anhydrous antiperspirant and/or deodorant compositions in the form of wax sticks. Gel sticks typically include water and upwards of 20% by weight of one or more polyhydric alcohols, e.g., glycols. Given the compositional differences between anhydrous compositions and gel sticks, it is not surprising that encaps commonly used in anhydrous compositions, e.g., starch, cyclodextrin or gelatin, fail to provide the same benefits in gel sticks.

Water-soluble encapsulating materials have the potential of being solubilized during production of gel sticks. Additionally, polyhydric alcohols employed in gel sticks, typically at relatively high levels, tend to be excellent solvents for most fragrances. Even if an encap is not-water soluble, minimizing fragrance extraction, either during production or in storage, can still be problematic in the case of polyhydric alcohol-containing gel compositions.

PCT Application No. PCT/EP2011/061782, filed Jul. 11, 2011, describes a class of encapsulates having a shell comprising what is therein referred to as a step-growth polymer and, interior thereto, a core comprising what is referred to as a chain-growth polymer. The described encaps are desirably produced by an emulsion polymerization, preferably interfacial polymerization, in which the mean particle diameter of the dispersed phase is very small, giving rise to particles of relatively small average diameter, including particles known as as mini-emulsion encaps or MEEs.

The shell of the particles described by PCT/EP2011/061782 is relatively thin in relation to the core and is generally porous to fragrance molecules. The encaps can be made as fragrance-free “blank(s)” that can be added as such, i.e., without fragrance, to a cosmetic formulation. Free fragrance incorporated into the cosmetic formulation partitions into the core of the blank, creating the loaded encap in situ. In the presence of solvents such as polyhydric alcohols, the porosity of the fragrance delivery particles can, however, aid in fragrance leaching.

While chemically more compatible with gel sticks than many of the encaps commonly employed in anhydrous compositions, the particles described by PCT/EP2011/061782 can vary widely with respect to their performance in such applications. Undesirable performance is often the result of particle flocculation. Flocculation can detract from the uniformity and or appearance of a product giving rise, for example, to haziness or reduced translucency and/or transparency. In the extreme, flocculation can also impair fragrance delivery. Additionally the potential remains for solvent extraction by the polyhydric alcohol, that is to say, with such particles, the longevity of product release can be inconsistent.

One aspect of this invention is to extend fragrance release, over time, of a polyhydric alcohol-containing gel. Another aspect of this invention is to provide a gel stick having a desirable visual appearance which composition includes both fragrance encap and a relatively high level of polyhydric alcohol. Yet another aspect of this invention is to minimize encap flocculation in a polyhydric alcohol-containing gel stick.

### SUMMARY OF THE INVENTION

It has been found that by the selection of encaps having certain compositional parameters, one or more aspects of this invention can be achieved. In one embodiment this invention relates to a gel composition which comprises:
(a) from 20 to 80% by weight of polyhydric alcohol,
(b) from 15 to 70% by weight of water,
(c) gelling agent, preferably comprising at least one alkali metal salt of fatty acid,
(d) fragrance-delivery particles comprising:
   i) at least one shell formed by a step-growth polymerization reaction,
   ii) interior said shell, a core comprising at least one region formed by chain-grown polymerization of one or more chain growth monomers selected from the group consisting of aryl methacrylate alkyl methacrylate, and mixtures thereof, wherein the alkyl group of said alkyl methacrylate is C₆ to C₂₀ alkyl, preferably C₈ to C₁₆ alkyl, and wherein the aryl group of said aryl methacrylate is of the formula —(CH₂)ₘ—Ar where m is an integer having a value of 0 to 4, preferably 0 or 1, most preferably 1, and Ar is phenyl, optionally substituted with up to three pendant alkyl groups, with the proviso that the total number of carbon atoms in the pendant alkyl groups combined does not exceed 10,
   iii) optionally, surfactant grafted to said shell, wherein the surfactant preferably comprises non-ionic surfactant with polyglycol functionalized chains, more preferably, polyethylene glycol-polypropylene glycol-polyethylene glycol block copolymer, wherein the fragrance delivery particles have an average diameter of less than 20 microns, and
(e) free fragrance,
wherein the composition is in the form of a deodorant stick.

In a further embodiment this invention relates to a process for producing a gel composition that comprises the steps of:
a) forming an aqueous dispersion of particles comprising:
   i) at least one shell formed by a step-growth polymerization reaction,
   ii) interior said shell a core comprising at least one region formed by chain-grown polymerization of one or more chain growth monomers selected from the group consisting of aryl methacrylate, alkyl methacrylate, and mixtures thereof, wherein the alkyl of said methacrylate is C₆ to C₂₀ alkyl, preferably C₈ to C₁₆ alkyl, and wherein the aryl group of said aryl methacrylate is of the formula —(CH₂)ₘ—Ar where is an integer having a value of 0 to 4, preferably 0 or 1, most preferably 1, and Ar is phenyl, optionally substituted with up to three pendant alkyl groups, with the proviso that the total number of carbon atoms in the pendant alkyl groups combined, does not exceed 10,
   iii) optionally, surfactant grafted to said shell, wherein the surfactant comprises non-ionic surfactant having polyglycol functionalized chains, more preferably, polyethylene glycol-polypropylene glycol-polyethylene glycol block copolymer,
wherein the particles comprise at least 5%, preferably 10% by weight of the dispersion, have an average diameter of less than 20 microns and are formed as fragrance free blanks,
b) forming a deodorant base comprising: water, polyhydric alcohol, preferably glycol, and gelling agent,
c) combining the aqueous particle dispersion, deodorant base, and fragrance, fragrance optionally being added as part of the deodorant base, and
d) allowing fragrance to partition into the blanks and the resulting gel composition to solidify.

### DETAILED DESCRIPTION OF THE INVENTION

As used herein, unless otherwise noted, all parts and percentages are by weight and are based on the entire weight of the composition. Except in the operating and comparative examples, or where otherwise explicitly indicated, all numbers in this description indicating amounts of material ought to be understood as modified by the word “about”. As used herein “essentially free of antiperspirant salts containing aluminum and/or zirconium” means that the total level of any such salts is too low to noticeably affect the clarity of the composition, which amount is typically an amount of 0.1% by weight of the composition or less. It is further noted that the use of “clear” in reference to the gelled compositions of the subject invention means that the composition is “transparent” or “translucent”. The term “transparent” as used in this specification is intended to connote its usual dictionary definition. Thus, a transparent cosmetic stick, like glass, allows for ready viewing of objects behind it. By contrast, a translucent cosmetic stick, although allowing light to pass through the stick, causes the light to be scattered so that objects behind the translucent stick are less clearly identified.

Within the context of this invention, a cosmetic composition is deemed to be “clear” if the transmittance of light of any wavelength in the range of 400 to 900 nm through a sample 1 cm thick is greater than 0.5% and, for transparent sticks, greater than 35%. Light transmittance can be easily measured by placing a stick sample of the required thickness into the light beam path of a UV-VIS spectrophotometer such as a Baush & Lomb Spectrophotometer.

Where compositions of the subject invention are described as “including” or “comprising” specific compositions or materials, narrower embodiments where the compositions can “consist essentially of” or “consist of” the recited components or materials are also contemplated.

### Carrier

The gel compositions of the subject invention are formed by the gelation of a water/polyhydric alcohol carrier. Water employed as part of the carrier is desirably de-ionized and, if desired, may be distilled. Deionization removes impurities which can interfere with gellation and/or form precipitates that can affect clarity. Desirably, water is present in the gelled compositions of this invention in an amount of at from 15 to 70% by weight, more particularly, from 15 to 50% by weight, and, in at least one embodiment of interest, from 20 to 35% by weight.

The carrier also includes one or more cosmetically acceptable polyhydric alcohols such as are conventionally employed in gel compositions. The cosmetically acceptable polyhydric alcohols are liquid at 20° C., and are miscible, preferably fully miscible, with water. In addition to functioning as part of the carrier, the polyhydric alcohols typically provide an emollient benefit to the compositions. Cosmetically acceptable polyhydric alcohols containing from 2 to 6, preferably from 2 to 3, hydroxyl groups are of particular interest. Examples of polyhydric alcohol suitable for use herein, include, for example, 1,2- and 1,3-propylene glycol, dipropylene glycol, 1,2-, 1,3- and 1,4-butylene glycol, glycerin, sorbitol, 1,2-pentylene glycol, 1,2-hexylene glycol, polyethylene glycol (for example, PEG 8, PEG 200 and PEG 400), and the like, and mixtures thereof, with a combination of propylene glycol (1,2- and/or 1,3-) and dipropylene glycol being of particular interest.

The total amount of polyhydric alcohol may range from about 20 to about 80% by weight of the gel composition, more particularly from about 30 to about 75% by weight of the composition and, in an embodiment of particular interest, from 40 to 70% by weight of the composition.

Desirably, the water and polyhydric alcohol-containing carrier comprises at least 75% by weight, preferably at least 80% by weight of the total composition. Compositions that include from 80 to 95% by weight of carrier are of particular interest

If desired, at least a portion of the carrier may comprise one or more cosmetically acceptable organic solvents that are liquid at 0° C. and miscible with the water and polyhydric alcohol mixture, and that do not undesirably effect gel formation and sensory properties of the resultant composition. Ethanol is among the organic solvents that may be so employed. Organic solvent may be desirable as an aid in “lifting” fragrance. Emollients other than polyhydric alcohol may also be employed as additional optional components of the carrier, provided that such emollients do not undesirably effect gel formation and sensory properties of the resultant composition. Exemplary of such additional optional emollients are PPG3-myristyl ether, isostearyl alcohol, and sunflower seed oil. When present, the total amount of such additional emollients desirably does not exceed 5% by weight of the gel composition.

### Gelling Agent

The gelling agent employed herein preferably comprises an alkali metal salt of a fatty acid (herein also referred to as a “soap”), preferably a C₁₂ to C₂₄ fatty acid, more preferably a C₁₆ to C₂₂ fatty acid, with sodium and potassium salts being among the preferred alkali metal salts. Included among the fatty acids from which such salts are derived, include, for example, coconut oil, beef tallow, lanolin, fish oil, beeswax, palm oil, peanut oil, olive oil, cottonseed oil, soybean oil, corn oil, rapeseed, rosin acids and greases. In at least one embodiment of interest the alkali metal salts of fatty acids are selected from sodium stearate, potassium stearate, potassium palmitate, sodium palmitate, and mixtures thereof.

The gelling agent may be added in salt form, or the salt may be generated in situ, such as, for example, by the reaction of the corresponding fatty acid and, for example, an alkali metal hydroxide. The gelling agent associates with water to form a gel network that provides structure to the composition. As fatty acids can potentially contribute to the stick having a hazy appearance, it is often preferable to add the salt form of the fatty acid, rather than generating the alkali metal salt of the fatty acid in situ. Further, given that alkali metal salts can break down to their fatty acids, to shift the equilibrium between the alkali metal salts and their corresponding fatty acids and alkali metal hydroxide toward the salt form of the fatty acid, it is often desirable for the composition to be formulated to a pH of from about 8 to about 10.5, preferably from about 9 to about 10.

In addition to the alkali metal salt of a fatty acid, the gelling agent may further comprise one or more co-gellants. The use of co-gellant can provide a clarity stabilizing effect upon the composition. By replacing a portion of the fatty acid salt with a co-gellant, formulators may lessen the potential for fatty acid formation and, further, may provide compositions that are milder to the skin.

Included among the co-gellants suitable for use in the subject composition are polyethylene oxide-polypropylene oxide block copolymers of the general structure:

R_{f}[(C₂H₄O)ₐ(C₃H₆O)_{b}(C₂H₄O)_{c}(C₃H₆O)_{d}]ₑ[H]_{g} (I)

wherein:
R is independently selected from hydrogen, hydroxyl, C₁₀-C₂₂ fatty alkoxide, and ethylene diamine units;
a, b, c and d are independently selected integers ranging from 0 to 200 with the proviso that the sum of a, b, c and d is at least about 50;
e is an integer from 1 to 4;
f is an integer from 0 to 1; and
g is an integer from 0 to 4.

Included among such co-gellants are poly(ethylene oxide)(propylene oxide) (ethylene oxide) block copolymers commonly known as poloxamers. Typical of this substance are a series of products from BASF Corporation sold under the Pluronic® trademark. In at least one embodiment of interest, copolymers of this type will have an average molecular weight ranging from about 5000 to about 50,000, preferably between about 6,000 and 15,000. As a co-gellant, melt/pour points of these materials should be at least 30° C., and optimally are at least 55° C. Illustrative commercially available poloxamers are Pluronic® F 127 block copolymer copolymers and Pluronic® F 108 block copolymers.

In the Formula I block copolymers, when f is 1 and R is an ethylene diamine unit, the general structure defines a tetra-functional copolymer derived from the sequential addition of propylene oxide and ethylene oxide to ethylene diamine and that may be described in terms of structures (II) and (III) as follows:

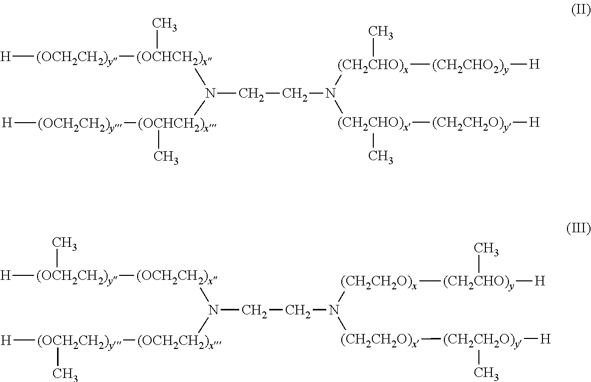

wherein X, X′, X″, X′″, Y, Y′, Y″, and Y′″ are integers such that the average molecular weight of the copolymer ranges from about 1,500 to about 100,000. Preferably, the average molecular weight should range from about 5,000 up to about 50,000, optimally between about 15,000 to about 30,000. In at least one embodiment of interest, these tetra-functional poloxamines are characterized as having a hydrophile-lipophile balance (HLB) of at least 12, preferably at least 18. HLB is an indicator of the relative attraction of a material for oil or water. Materials that are more oil soluble have lower HLB values, while materials that are more water soluble have higher HLB values. A method for determining HLB value is described by J. T. Davies in the Proceedings of the International Congress of Surface Activity (1957), pp. 426-438, which method is based on a material's molecular structure. As a cogellant, the poloxamines desirably have a melt/pour point of at least 30° C., preferably greater than 40° C., and in at least one embodiment of interest, greater than 50° C. Suitable poloxamines are commercially available from BASF Corporation under the trademark, Tetronic®. Especially suitable are the Tetronic® 1107, Tetronic® 1307 and Tetronic 1508® block copolymers.

Desirably, the total amount of gelling agent is present in the subject composition in an amount sufficient to provide the composition with a self-supporting structure, however, the selection and amount of gelling agent should not undesirably detract from the clarity desired in the ultimate product In the practice of this invention, total amounts of gelling agent of from 1 to 25% by weight of the composition, preferably from 3 to 20% by weight of the composition, more preferably 3 to 15% by weight of the composition are of particular interest. Ranges of preference depend, in part, on the choice and relative amounts of the gelling agent components, as well as the hardness and clarity desired in the gelled stick. In one embodiment of interest, when co-gellant is present, the total amount of the alkali metal salt of a fatty acid is desirably from 2 to 15% by weight of the composition, with amounts of from 3 to 10% by weight of the composition being of particular interest. When co-gellant is absent, the level of alkali metal salt of a fatty acid typically employed is from 5 to 25% by weight of the composition, more particularly from 5 to 20% by weight of the composition, and, in at least one embodiment, from 5 to 15% by weight of the composition. Desirably, the go-gellant is present in the subject compositions in an amount up to 6% by weight, preferably from 0.5 to about 5% by weight of the composition. In one embodiment of particular interest, the compositions of interest contain from 5 to 10% by weight of alkali metal salt of fatty acid and, as a co-gellant, from 1 to 4% by weight of Formula I block copolymer.

Waxes and other organic or inorganic opacifying materials that, if present, otherwise detract from the clarity desired in the gel stick, should be kept to a level that does not undesirably interfere with stick clarity. Desirably, the total amount of such opacifying materials, should not exceed 1% by weight of the composition; preferably such opacifying materials should be absent or not exceed 0.5% by weight of the composition.

### Fragrance Delivery Particles

The fragrance delivery particles of the subject compositions are characterized as comprising at least one shell formed by a step-growth polymerization reaction and at least one region formed by chain-grown polymerization. The particles belong to a class of encapsulates described in further detail in PCT/EP2011/061782, incorporated herein by reference. The interior region or “core” provides a sink for fragrance and, optionally, other benefits agents that formulators elect to encapsulate. The “shell” functions primarily to retain and protect the core. The structure of the subject particles allows for the late-stage addition of fragrance, as the particles and the fragrance may be dosed into the product separately.

As in PCT/EP2011/061782, in the present specification a distinction will be drawn between step-growth and chain-growth polymerization. This is the well-established reaction mechanism distinction drawn by Paul Flory in 1953 (see Paul J. Flory, “Principles of Polymer Chemistry”, Cornell University Press, 1953, p. 39. ISBN 0801401348).

For the purposes of the present specification a chain-growth polymer is a polymer formed by a reaction in which monomers, in the case of the subject invention particular alkyl- and/or arylmethacrylates, bond together via rearrangement without eliminating other molecules molecules in the process. Chain-growth polymers grow in a single direction from one end of the chain only and an initiator is typically used. In chain-growth polymerization it is commonplace that once a growth at a chain end is terminated the end becomes unreactive.

A step-growth polymer is a polymer whose chain is formed during the reaction of poly-functional monomers to form increasingly larger oligomers. Growth occurs throughout the matrix and the monomer level falls rapidly in the early stages of the reaction. No initiator is needed for a step growth polymerization and the ends of the growing chain generally remain active at all times. Typically (but not always) a small molecule, which is often water, is eliminated in the polymerization process.

An example of step-growth polymerization is the formation of polyester by the reaction of dicarboxylic acids and glycols with elimination of water. Another example is the polymerization of phenol and formaldehyde to produce “Bakelite”. Other well known step-growth polymerization reactions are the formation of polyesters, polyurethanes, polyureas, polyamides and polyethers.

The step-growth polymerization reaction that forms the shell of the subject fragrance delivery particles is desirably carried out as an interfacial polymerization. The step-growth polymerization reaction preferably involves an isocyanate monomer, more preferably a urethane and/or a urea. Isocyanate monomers are reactive, enable high monomer conversion, and form a robust, glassy shell which can survive drying and other processing. Isocyanate monomers react by a step-growth mechanism but are categorized as an addition polymer by virtue of no small molecule being eliminated during polymerization.

The chain growth polymerization by which the interior region of the subject particles is formed employs one or more monomers selected from the group consisting of aryl methacrylate alkyl methacrylate, and mixtures thereof, wherein the alkyl group of said alkyl methacrylate is C₆ to C₂₀ alkyl, preferably C₈ to C₁₆ alkyl and wherein the aryl group of said aryl methacrylate is of the formula —(CH₂)ₘ—Ar where m is an integer having a value of 0 to 4, preferably 0 or 1, most preferably 1, and Ar is phenyl, optionally substituted with up to three pendant alkyl groups, with the proviso that the total number of carbon atoms in the pendant alkyl groups combined does not exceed 10. Preferably the aryl methacrylate is benzyl methacrylate the phenyl group of which is optionally substituted by up to three pendant groups independently selected from linear or branched alkyl groups, wherein the pendant groups in combination provide a total of up to 10 carbon atoms. In one ore more embodiments of interest, the aryl methacrylate is unsubstitued.

In the practice of the subject invention the methacylates are preferably selected to such that the core polymers produced have solubility parameters that are similar to or match those of fragrance components commonly used in deodorant formulations. Accordingly the methacrylates are preferably selected to provide core polymers having solubility parameters of between 17 and 19.5, inclusive, as calculated by the method of S W Van Krevelen in Properties of Polymers p 200-225, Elsevier, (1990).

Optimizing the matching between the solubility parameters of the fragrance and the methacrylate comprising the inner region aids in achieving absorption of fragrance into the particles and/or in extending the life of the particles with respect to fragrance delivery.

Most preferably the methacrylates are selected from C₈ to C₁₆ alkyl methacrylate and/or optionally substituted benzyl methacrylate monomers, with a combination of such alkyl methacrylate and benzyl methacrylate monomers being of particular interest in one or more embodiments.

Advantageously the particle shell is surface grafted with surfactant. Surface modification has been found to aid in reducing particle flocculation. Surfactants of particular interest include nonionic surfactants and most particularly those with polyglycol functionalised chains. Particularly suitable surfactants include polyethylene glycol-polypropylene glycol-polyethylene glycol copolymers, especially polyoxyethylene glycol-polypropylene glycol-polyethylene glycol block copolymers broadly described above in connection with co-gellants, in particular, block copolymers known as poloxamers and poloxamines preferably having molecular weights of from 2,000 to 15,000, more particularly from 3,000 to 6,000. In one or more embodiments, polyoxomers are of particular interest.

The fragrance delivery particles employed herein (alternatively referred to as fragrance encaps or simply encaps), inclusive of surface modifying surfactant, have an average diameter of less than 20 microns, preferably less than 10 microns. In one or more embodiments particles having an average diameter of less than 1 micron are of particular interest. One benefit of small particles is that they tend to remain better suspended during product gellation. Particles having an average diameter of from 0.5 to 1 micron are of interest in at least one embodiment.

Fragrance delivery particles according to the present invention may be formed from an emulsion by carrying out an interfacial step-growth polymerization first to form a shell under conditions where the chain-growth polymerization is inhibited. Subsequently, the conditions are changed such that the material within the shell undergoes the chain-growth polymerization. A suitable change in conditions is to increase the temperature from one at which the chain growth reaction is inhibited to one at which it proceeds. Other possible changes of conditions would be, for example, to use a chain-growth reaction which is light dependent rather than temperature dependent.

The particles can be formed in the presence of the fragrance to be encapsulated by emulsion polymerization. In one such method an emulsion is prepared having as the dispersed phase, a non-aqueous phase, preferably having a mean dispersed particle size diameter of less than 1000 nm in which is dispersed phase is contained both a step growth monomer, preferably isocyanate, for the step step-growth polymerization, chain-growth monomer(s) (i.e., the aryl- and or alkylmethacrylate), fragrance to be encapsulated, and a radical initiator for the chain growth polymerisation of the methacrylates. The continuous phase of the emulsion is an aqueous phase and includes water and a co-monomer for the step-growth polymerization, preferably a diol or diamine. The emulsion is maintained under conditions, e.g., temperature, at which the step-growth polymerization occurs, but not the chain growth polymerization, and, thereafter is maintained at conditions, e.g., temperature, photokinetics, and/or the like, at which the chain-growth polymerization proceeds.

Preferably the step growth monomer and co-monomer react by a step-growth mechanism to form a poly-urethane (which may be illustrated by the approximate formula (—R₁—NH—CO—O—R₂—O—CO—NH—)ₙ) or a polyurea (which may be illustrated by the approximate general formula (—NH—CO—NH—R—)ₙ).

The monomer capable of chain-growth polymerization is preferably ethylenically unsaturated, more preferably vinyllic. In the alternative, a ring-opening mechanism may be used.

Advantageously, the above described method provides a potentially “one-pot” reaction which has the advantages of simplicity and reduced losses: i.e. the shell is formed by step-growth polymerization at the interface of the emulsion droplets and the core is subsequently formed within the shell by an in-situ chain-growth polymerization.

Fragrance delivery particles are alternatively, and in one or more embodiments, more desirably prepared in situ by a process as described above in which fragrance is omitted, and the particles prepared as fragrance-free “blanks”. The fragrance-free particles and fragrance may be added separately to the gel formation and the blank encaps loaded in situ by partitioning of the free fragrance into the formulation. Loading in situ offers the advantage of allowing a single encapsulate to be used with a range of free fragrances with minimal risk of fragrance distortion caused by mixing of different fragrances.

Irrespective of whether fragrance is pre-loaded into the fragrance delivery particles or is loaded in situ, partioning of the fragrance will result in free oil being present in the gel composition.

Especially suitable step-growth polymers are those whose isocyanate monomers are aromatic polyisocyanates, aliphatic polyisocyanates, and mixtures thereof.

Suitable aromatic polyisocyanates comprise, but are not limited to, 2,4- and 2,6-toluene diisocyanate, naphthalene diisocyanate, diphenyl methane diisocyanate and triphenyl methane-p,p′p″-trityl triisocyanate, polymethylene polyphenylene isocyanate, 2,4,4′-diphenylether triisocyanate, 3,3′-dimethyl-4,4′-diphenyl diisocyanate, 3,3′-dimethoxy-4,4′diphenyl diisocyanate, and 4,4′4″-triphenylmethane triisocyanate.

Suitable aliphatic polyisocyanates comprise, but are not limited to dicyclohexylmethane 4,4′-diisocyanate, hexamethylene 1,6-diisocyanate, isophorone diisocyanate, tri methyl-hexamethylene diisocyanate, trimer of hexamethylenel,6-diisocyanate, trimer of isophorone diisocyanate, 1,4-cyclohexane diisocyanate, urea of hexamethylene diisocyanate, trimethylene diisocyanate, propylene-1,2-diisocyanate and butylenel,2-diisocyanate and mixtures thereof.

In one or more embodiments, preferred isocyanate materials are 2,4- and 2,6-toluene diisocyanate, and isophorone diisocyanate, with isophorone diioscyanate being of particular interest in one or more embodiments.

The co-monomer used in the step-growth polymerization is typically a diol or a diamine.

Suitable diols can comprise, but are not limited to, low molecular weight polymers such as ethylene glycol, diethylene glycol, propylene glycol, 1,4-butanediol, 2,3-butane diol, neopentyl glycol, 1,6-hexanediol, dipropylene glycol, cyclohexyll,4-dimethanol, 1,8-octanediol; high molecular weight polyols such as polyethylene glycol, polypropylene glycols, polytetramethylene glycols (PTMG) having average molecular weight in the range of 200 to 2000, polyester diols, diols containing carboxyl groups such as dimethylol propionic acid (DMPA) and dimethylol butanoic acid (DMBA) and mixtures thereof.

The preferred diol materials are ethylene glycol, diethylene glycol, propylene glycol, 1,4-butanediol, 2,3-butane diol, neopentyl glycol, 1,6-hexanediol, and dipropylene glycol. The more hydrophobic diols (particularly 1,4-butanediol, 2,3-butane diol, neopentyl glycol and 1,6-hexanediol) are preferred as it is generally easier to get a stable emulsion with these materials and thereby a more efficient polymerization.

Suitable diamines can comprise amines such as ethylene diamine (EDA), phenylene diamine, toluene diamine, hexamethylene diamine, diethylenetriamine, tetraethylene pentaamine, pentamethylene hexamine, 1,6-hexane diamine, Methylene tetramine, 2,4-diamino-6-methyl-1,3,5 triazine 1,2-diaminocyclohexane, 4,4′-diamino-diphenylmethane, 1,5-diaminonaphthalene, 2,4,4′-triaminodiphenylether, bis(hexa-methylenetriamine), 1,4,5,8-tetraaminoanthraquinone, isophorone diamine, diamino propane and diaminobutane, and mixtures thereof.

The preferred diamine materials are ethylene diamine and 1,6-hexane diamine. Mole ratios of the co-monomers are preferably selected such that the water soluble monomer is present in up to 10 mol % excess over the oil soluble co-monomer, preferably 1 to 8 mol % excess, more preferable 2 to 5 mol % excess. It is believed that this ensures complete reaction of isocyanate monomer.

Conveniently the fragrance delivery particle further comprises a cross-linking agent, derived from a more than di-functional species having isocyanate, alcohol, amine functionality, and/or a more than mono-functional vinyllic monomer. Tri- and tetra-functional materials are preferred. The benefit of cross-linking agents is to increase robustness of either the shell or the inner region, and or decrease permeability. Cross-linking agents in the inner region can modify interaction of the “core” with the fragrance, e.g. by modification of the solubility parameters.

Many cross-linking agents suitable for use in step-growth polymerization are known. Among such cross-linking agents are polyamines and polyols.

Preferred amine-functional cross-linking agents contain more than two amine functionalities such as tetraethylene pentamine, triethylene tetraamine, 2,4,4′-triaminodiphenylether, bis(hexamethylene triamine), 1,4,5,8-tetraamino anthraquinone and diethylene triamine (DETA), and mixtures thereof.

Preferred alcohol-functional cross-linking agents contain more than two alcohol functionalities such as glycerol, pentaerythritol, and 1,1,1 trihydroxmethylpropane.

A particularly preferred cross-linking agent is polyphenylisocyanate.

The preferred levels of cross-linking agent are 1-50 mol %, more preferably 2-35 mol % of the shell-forming monomers.

As noted above at least one region interior to the shell is formed by chain-growth polymerization. Typically this will comprise a single solid region making-up the “core” of the particle.

Free-radical polymerization (FRP) is a suitable method of chain-growth polymerization. In FRP a mono-functional monomer is polymerised in the presence of free-radical initiator

The free-radical initiator can be any molecule known to initiate free-radical polymerization such as azo-containing molecules, persulfates, redox initiators, peroxides, benzyl ketones. These initiators may be activated via thermal, photolytic or chemical means, with thermal activation being of particular interest.

Examples of suitable initiators include but are not limited to 2,2′-azobisisobutyronitrile (AIBN), azobis(4-cyanovaleric acid), benzoyl peroxide, cumylperoxide, 1-hydroxy-cyclohexyl phenyl ketone, t-butyl hydroperoxide, and ascorbic acid.

In some cases, more than one initiator may be used.

The preferred initiators are: 2,2′-Azobis(2-methylbutyro-nitrile), 2,2′-Azobis(2.4-dimethyl valeronitrile), 1,1′-Azobis(cyclohexane-1-carbonitrile) and t-butyl hydroperoxide/ascorbic acid as these minimise the production of unwanted bi-products.

Preferably, the residue of the initiator in a free-radical polymerization comprises 0 to 5% w/w, preferably 0.01 to 5% w/w and especially 0.01 to 3% w/w, of the resulting copolymer based on the total weight of the monomers.

The weight fraction of step growth polymer in the combined step growth and chain growth polymers comprising the particle is typically 1% to 99%, more particularly 2% to 80%, even more particularly from 5% to 75%. In one or more embodiments of interest the weight fraction of step growth polymer in the combined step growth and chain growth polymers comprises from 1 to 25%, more particularly from 2 to 20%.

Cross-linking agents, as distinguished from chain growth monomer, can be used to modify the properties of the chain-growth polymer. Suitable materials comprise a molecule containing at least two vinyl groups that may be polymerised. The molecule may be hydrophilic, hydrophobic, amphiphilic, neutral, cationic, zwitterionic or oligomeric. Examples include di- or multivinyl esters, di- or multivinyl amides, di- or multivinyl aryl compounds and di- or multivinyl alk/aryl ethers. Typically, in the case of oligomeric or multifunctional branching agents, a linking reaction is used to attach a polymerisable moiety to a di- or multifunctional oligomer or a di- or multifunctional group. The brancher may itself have more than one branching point, such as ‘T’-shaped divinylic oligomers. In some cases, more than one multifunctional monomer may be used.

Macro cross-linkers or macro branchers (multifunctional monomers typically having a molecular weight of at least 1000 Daltons) are generally formed by linking a polymerisable moiety, such as a vinyl or aryl group, to a pre-formed multifunctional polymer via a suitable linking unit such as an ester, an amide or an ether. Examples of suitable polymers include di-functional poly(alkylene oxides) such as poly(ethyleneglycol) or poly(propylene glycol), silicones such as poly(dimethyl-siloxane)s, polymers formed by ring-opening polymerization such as poly(caprolactone) or poly(caprolactam) or poly-functional polymers formed via living polymerization such as poly(1,4-butadiene).

Preferred macro branchers include poly(ethyleneglycol)di(meth)acrylate, poly(propyleneglycol)di(meth)acrylate, (meth)acryloxypropyl-terminated poly(dimethylsiloxane), poly(caprolactone)di(meth)acrylate and poly(caprolactam)di(meth)acrylamide.

The corresponding allyl monomers to those listed above can also be used where appropriate.

Preferred multifunctional monomers include but are not limited to divinyl aryl monomers such as divinyl benzene; (meth)acrylate diesters such as glycerol di(meth)acrylate, ethylene glycol di(meth)acrylate, propyleneglycol di(meth)acrylate and 1,3-butylenedi(meth)acrylate; oligoalkylene oxide di(meth)acrylates such as tetra ethyleneglycol di(meth)acrylate, oligo(ethyleneglycol)di(meth)acrylate and oligo(propyleneglycol)di(meth)-acrylate; divinyl acrylamides such as methylene bis-acrylamide; silicone-containing divinyl esters or amides such as (meth)acryloxypropyl-terminated oligo (dimethyl-siloxane); divinyl ethers such as oligo (ethyleneglycol)-divinyl ether; and tetra- or tri-(meth)acrylate esters such as pentaerythritol tetra-(meth)acrylate, trimethylolpropane tri(meth)acrylate or glucose di- to penta(meth)acrylate. Further examples include vinyl or allyl esters, amides or ethers of pre-formed oligomers formed via ring-opening polymerization such as oligo(caprolactam) or oligo-(caprolactone), or oligomers formed via a living polymerization technique such as oligo(1,4-butadiene).

Especially preferred cross-linkers are divinyl benzene, ethylene glycol di(meth)acrylate and trimethylolpropane tri(meth)acrylate.

Levels of cross-linker are typically 0-75, preferably 0.0001 to 50, more preferably 0.0001 to 25 mol % of the core-forming.

Whether loaded in situ or incorporated into the fragrance delivery particles prior to their addition to the subject gel compositions, fragrance is or becomes a part of such particles. As used herein the term “fragrance” extends to perfume, pro-fragrance, and other fragrance components. The perfume suitably has a molecular weight of from 50 to 500 Dalton. Pro-fragrances can be of higher molecular weight, being typically 1-10 kD.

Fragrance is typically present in an amount of from 5-85% by total weight of the fragrance delivery particle, preferably from 10 to 75% by total weight of the core-forming monomers.

Useful components of the fragrance include materials of both natural and synthetic origin. They include single compounds and mixtures. Specific examples of such components may be found in the current literature, e.g., in Fenaroli's Handbook of Flavour Ingredients, 1975, CRC Press; Synthetic Food Adjuncts, 1947 by M. B. Jacobs, edited by Van Nostrand; or Perfume and Flavour Chemicals by S. Arctander 1969, Montclair, N.J. (USA). These substances are well known to the person skilled in the art of perfuming and/or aromatizing consumer products, i.e., of imparting an odour to a consumer product that is traditionally perfumed, or of modifying the odour of said consumer product.

In addition to fragrance, the subject fragrance delivery particles may optionally include one or more hydrophobic benefit agents, preferably an organoleptic benefit agent. Such additional benefit agents include, for example, dyes, pigments and/or other colorants, sunscreens, skin lightening agents, ceramides, antioxidants, and antimicrobial agents.

### Encap Preparation Methods:

Polymerization occurs in at least two phases. In an earlier of these phases a shell is formed by a step-growth polymerization. This shell encloses and contains the reagents for the chain-growth reaction which occurs in a later phase.

Temporal separation of these phases is accomplished by control of the reagents present and the reaction conditions.

Typically, at least one of the components of the shell-forming reaction is withheld from the initial reaction mixture and added gradually to control the progress of the reaction in the first phase.

Advantageously, the first phase of the reaction is performed under conditions in which the chain-growth reaction is inhibited. These conditions include a sufficiently low temperature (for a thermally activated reaction) or conditions of sufficiently low light (for a photo-activated reaction).

Once the shell-forming reaction has proceeded sufficiently, the conditions are modified (for example, by raising the temperature or exposing the reaction mixture to light) to cause the reaction to form the inner region to start.

The preferred method is one in which an emulsion is formed comprising the chain-growth polymer components in a non-aqueous dispersed phase and the step-growth polymer components are at the interface between the dispersed phase and the continuous aqueous phase.

Typically the aqueous phase comprises an emulsifying agent, and one of the co-monomers for the step-growth polymer. It may also contain any diol, alcohol or amine cross-linking agent.

The disperse phase comprises the chain-growth monomer, the initiator, any isocyanate or vinyl cross-linking agents, the other co-monomer for the step growth polymer and any optional benefit agent.

Fragrance may be present in the reaction mixture, at a level to give the resulting particles fragrance at the levels disclosed above, although it is also possible, and in one or more embodiments preferable, to form “empty” particles and subsequently expose them to fragrance which can be adsorbed into the inner region. Thus, the particles of the present invention are particularly suited to processes for manufacture of products which feature “late variant addition” of fragrance and, optionally, other benefit agents.

Surfactants employed as surface modification materials are generally added to the aqueous phase towards the end of the process, where, for example, further monomer(s) can be added to form further shell material and bind additional materials to the outside of the particle.

Many emulsifying agents are known for use in emulsion polymerization. Suitable emulsifying agents for use in the polymerization process may comprise, but are not limited to, non-ionic surfactants such as polyvinylpyrrolidone (PVP), polyethylene glycol sorbitan monolaurate (Tween 20), polyethylene glycol sorbitan monopalmitate (tween 40), polyethylene glycol sorbitan monooleate (Tween 80), polyvinyl alcohol (PVA), and poly(ethoxy)nonyl phenol, ethylene maleic anhydride (EMA) copolymer, Easy-Sperse™ (from ISP Technologies Inc.), ionic surfactants such as partially neutralized salts of polyacrylic acids such as sodium or potassium polyacrylate or sodium or potassium polymethacrylate. Brij™-35, Hypermer™ A 60, or sodium lignosulphate, and mixtures thereof.

Emulsifiers may also include, but are not limited to, acrylic acid-alkyl acrylate copolymer, poly(acrylic acid), polyoxyalkylene sorbitan fatty esters, polyalkylene co-carboxy anhydrides, polyalkylene co-maleic anhydrides, poly(methyl vinyl ether-co-maleic anhydride), poly(propylene-co-maleic anhydride), poly(butadiene co-maleic anhydride), and poly(vinyl acetate-co-maleic anhydride), polyvinyl alcohols, polyalkylene glycols, polyoxyalkylene glycols, and mixtures thereof.

Preferred emulsifying agents are fatty alcohol exthoylates (particularly of the Brij™ class), salts of ether sulphates (including SLES), alkyl and alkaryl sulphonates and sulphates (including LAS and SDS) and cationic quaternary salts (including CTAC and CTAB).

It is particularly preferred that the emulsifying agent comprises a nonionic surfactant. It is also preferred that the non-ionic surfactant is hydrophilic, so as to promote the formation of a stable mini-emulsion. Preferably, the ratio of non-ionic to anionic emulsifier should be greater than 1:1 (i.e. non-ionic is present in excess) and the total surfactant level should be >3% wt of the polymerization mixture.

### Co-Surfactant:

Typically a co-surfactant will be present in the dispersed phase and in the resulting particle. Suitable co-surfactants for use in the present invention include hexadecane, cetyl alcohol, lauroyl peroxide, n-dodecyl mercaptan, dodecyl methacrylate, stearyl methacrylate, polystyrene, polydecene, mineral oils, isopropyl myristate C₁₂-C₁₅ alkyl benzoate and polymethyl methacrylate.

The preferred cosurfactants comprise hexadecane, polydecene and isopropyl myristate, C₁₂-C₁₅ alkyl benzoates.

As a wt % of oil phase as a total, the co-surfactant is typically 0-20%, preferably 1-15%, more preferably 2-12.5%.

The level of catalyst is typically 0.1-2% with respect to chain-growth monomer.

### Polymerization Conditions:

As noted above, polymerization typically occurs in at least two phases. In the earlier phase the shell is preferably formed by a reaction which preferably occurs at less than about 60 Celsius, more typically 15-55 Celsius. In the later phase the inner region is polymerised at a preferred temperature of more than about 70 Celcius, typically 70-95 Celcius.

Both reactions are allowed to proceed for sufficiently long for polymerization to be essentially complete, 1-3 hours being typical for each stage.

Surfactant may added at the end of the later phase (preferably after cooling), when for example, further shell forming material (for example further isocyanate and co-monomer) are also added to bind the surfactant to the outer surface of the particle by the formation of further shell material which entraps a portion of the surfactant and leads to a “hairy” particle in which the “hair” comprises the surfactant.

Preferably the emulsion polymerization step is a so-called “mini-emulsion” polymerization, performed with a dispersed phase droplet size of below one micron. Sufficiently fine emulsions can be obtained by a range of methods, including sonication, and/or via high shear dynamic mixers or static mixers. Mini-emulsion products have excellent suspending properties.

### Clarifying Agent

Optionally, the gel compositions of this invention may further comprise a clarifying agent for maintaining the clarity of the gelled composition. This agent is preferably a basic amine selected from amino alkanols having from 2 to 6 hydroxyl groups. These alkanols may have anywhere from 3 to 5 carbon atoms and have molecular weights less than 1,000. Particularly effective are the propanol amines. Illustrative of this category is tetra(hydroxypropyl)diamine, available from BASF Corporation under the trademark, QUADROL®. Another preferred amino alkanol is 2-amino-2-methylpropan-1-ol, available from the Angus Chemical Company under the trademark, AMP®, Also suitable are 2-amino-2-ethyl-1,3-propanediol, available from the Angus Chemical Company under the trademark, AMPD® and 2-amino-2-hydroxymethyl-1,3-propanediol (generically referred to as tromethamine), available from the Siga Chemical company.

The basic amine may be present in amounts up to 2% by weight of the composition, more particularly, from 0.05 to 1% by weight of the composition. In an embodiment of particular interest, the basic amine is present in the compositions of this invention in an amount of from about 0.1 to about 0.6% by weight of the composition.

### Deodorant Actives

In addition to the polyhydric alcohol, which may afford a deodorancy benefit in its own right, the gel compositions of the subject invention may further comprises other materials that provide a malodour-reducing or deodorancy benefit. Other materials that may be employed as deodorant actives are antimicrobial agents that inhibit microbial activity by bacteriostatic or inhibitatory activity, for example, the antimicrobial agents may inhibit the metabolic activity of odor generating microbes. Suitable deodorant actives include, but are not limited to, 2,2′-methylenebis(3,4,6,-trichlorophenol), 2,4,4′-trichloro-2′-hydroxydiphenylether (Triclosan), N-(4-chlorophenyl)-N′-(3,4-dichlorophenyl) urea (Triclocarban), 2,2′-thiobis(4,6-dichlorophenol), p-chloro-m-xylenol, dichloro-m-xylenol, polyaminopropyl biguanide, and the like. Quaternary ammonium compounds such as, for example, benzethonium chloride, cetyl pyridium chloride, cetyl pyridinium chloride, lauryl pyridium chloride, methylbenzethonium chloride, and cetyl-trimethyl ammonium bromide, and the like, may also be used in the subject compositions as antimicrobial agents. Other bacteriostats suitable for use herein are, for example, triethyl citrate, sodium N-lauroyl sarcosine, sodium N-palmitoyl sarcosine, and lauroyl sarcosine. The deodorant actives may be used alone or in combination. The amount of antimicrobial present in the compositions may range from about 0.001 to about 3% by weight of the composition, with ranges of depending upon the particular active employed. For example, when Tricolsan, available from Ciba-Geigy Corporation under the trademark, IRGASAN® DP-300, is utilized, the amount thereof is typically from 0.05 to about 1% by weight of the composition, more particularly from 0.1 to about 0.5% by weight of the composition.

### Other Components

Gel compositions of the invention may further comprise additional cosmetic ingredients including, but not limited to preservatives, colorants, sunscreen, chelating agents, pH adjusters, viscosity modifiers, and free fragrance. In at least one embodiment of interest, the total amount of such additional optional ingredients does not exceed 8 by weight of the gel compositions. In at least one embodiment, the total amount of such additional ingredients is from 0.01 to 5% by weight, more particularly preferably from 0.1 to 3% by weight of the gel compositions.

Desirably the gel compositions are essentially free of astringent antiperspirant salts containing aluminum and/or zirconium. In at least one embodiment of interest the compositions of interest contain less than 1% by weight of antiperspirant salts containing aluminum and/or zirconium.

As deodorant sticks it is preferred that the gelled compositions of the subject composition have a penetration hardness of 6-14 mm. As used herein the hardness of the subject composition refers to the hardness of the compositions in gelled form as measured using a penetrometer (PNT penetrometer equipped with a Seta wax needle (weight 2.5 grams) having a cone angle at the point of the needle of 9° 10′+/−15′). Sticks are allowed to equilibrate for at least 24 hours at a temperature of 22° C. prior to measurement. The needle is lowered into a flat upper surface of the composition and penetration hardness is measured by allowing the needle with its holder to drop under the combined weight of needle and holder (50 g) for a period of five seconds. The test is carried out at six points on each sample and the results then averaged.

### Preparation

The subject gel compositions can be prepared by conventional procedures. For example, the water and polyhydric alcohols may be combined and heated to temperatures of 70 to 90° C., the gelling agent, co-gellant and, if present, clarifying agent, are then introduced and dissolved. Once dissolution is complete the encap particle dispersion, fragrance loaded or blank, is added. The resulting blend is cooled to temperature about 5 to about 25° C. above the gelling temperature of the composition and any volatile or temperature sensitive ingredients, including free fragrance, are then introduced and the resulting composition is transferred to a conventional gel stick container at a suitable pour temperature, typically a temperature about 2 to about 5° C. above the gel temperature of the composition, and cooled to room temperature

In order that the present invention may be still further understood and carried forth into practice it will be further described with reference to the following non-limiting examples:

### Examples

### Fragrance Delivery Particle (Blanks) Formulation

Aqueous and oil phases, as well as a surfactant solution (for the first stage of the polymerization) and initiator solutions for the second stage free radical polymerization) were prepared as described below.

### Oil Phase

Poly(phenyl isocyanate) (Aldrich 372986)=3.04 g
Isophornone diisocyanate=6.35 g

### Methacrylate (Benzyl Methacrylate (Methacrylate A) or Lauryl Methacrylate

### (Methacrylate B)=12.67 g

### Dimethylethanolamine=0.0044 g

### Isopropyl Myristate (Crodamol™ IPM—Croda)

### Aqueous Phase

1,6-hexanediol=3.38 g
1,1,1-tri(hydroxymethyl) propane=1.20 g
Ethoxylated (20) fatty alcohol based on cetyl alcohol (Brij™ C20; Croda)=1.8 g
Sodium lauryl ether sulfate (1 EO), aka SLES (1 EO)=0.64 g

### Water=67.2 g

### Surfactant Solution

Polyoxyethylene-polyoxypropylene-polyoxyethylene block copolymer (Pluronic® P65 or P123 Block Copolymer Surfactant)=0.3 g

### Water=2.7 g

### Initiator 1

### Ascorbic Acid=0.12 g

### Water=1.2 g

### Initiator 2

Tert-Butyl hydroperoxide=0.25 g

### Water=5 g

### Brij™ C20=0.07 g

### SLES 1 EO=0.03 g

### Initiator 3

### Ascorbic Acid=0.05 g

### Water=0.5 g

### Method

1. Poly(phenyl isocyanate), isophorone diisocyanate, dimethylethanolamine, isopropyl myristate and the selected methacrylate were combined in a 30 ml vial.
2. SLES-1 EO, Brij C20, 1,1,1-tris(hydroxymethyl)propane and 1,6-hexane diol were dissolved in water and cooled to below 10° C.
3. Using a sonic probe the oil and aqueous phases were mixed while cooled in an ice bath. The oil and aqueous phases were mixed for two periods of 90 seconds each with the sample being shaken in between each period). The final temperature of the resulting mini-emulsion was approximately 25° C.
4. The mini-emulsion was placed in a 250 ml round bottom flask and stirred at a temperature of 50° C. and 200 rpm for twenty five minutes.
5. For the particles surface grafted with surfactant, the surfactant solution described above was added drop-wise over a two minute period and the reaction stirred for a further 150 minutes.
6. The temperature was increased to 75° C. and initiator solution 1 was added.
7. Initiator solution 2 was added vial peristaltic pump or syringe pump over 45 minutes.
8. The reaction mixture was stirred for a further 60 minutes.
9. Initiator 3 was added and the reaction mixture was stirred for a further 30 minutes.
10. After ˜5.5 hours the reaction was cooled and decanted.
11. The resulting encaps had a cross-linked polyurethane shell approximately 25 nm thick surrounding a polymetacrlate core. Those particles treated with the surfactant solution were surface grafted with the PEG-PPG-PEG poloxamer. The encaps had an average diameter of less than 1 um.

### Stick Formulation

Compositions having the formulation described below were formulated as deodorant sticks. As the general procedure for preparing the sticks, the propylene glycol, dipropylene glycol, water, sodium stearate and poloxamine were weighed into a beaker and heated with stirring using a Heidolph stirrer to 85° C. (˜260 rpm on stirrer). The mixture was cooled to 77° C., AMP was added, and the mixture further cooled to 75° C. at which point the blank fragrance delivery particles, prepared as described above, were pipetted in slowly. The mixture was cooled to 67° C. and fragrance was added. The mixture was then cooled to 55° C. and poured to form sticks. The sticks were cooled until solid and then allowed to stand at room temperature overnight before being assessed for flocculation.

TABLE 1
Stick Formulation

| Component | Wt.% |
|---|---|
| Propylene Glycol | 22.50 |
| Dipropylene Glycol | 40.00 |
| Sodium Stearate | 5.50 |
| Polyoxyethylene-Polyoxypropylene | 3.00 |
| Block Copolymer of Diethylamine | |
| (Tetronic ® 1307 Poloxamine; BASF) | |
| Water | 24.07 |
| 2-Amino-2-methylpropan-1-ol (AMP) | 0.40 |
| Aqueous dispersion of blank encap | 3.03 |
| particles (33% by wt. particles) | |
| Fragrance | 1.5 |
| Total | 100% |

The flocculation assessment was carried out with the aid of a high intensity light source (Schott KL150 LCD) shone directly through the stick sample and reported based on the following five point assessment scale:

| Degree of Flocculation | Score |
|---|---|
| No Floccs visible | 0 |
| Very fine floccs just visible | 1 |
| Fine floccs visible | 2 |
| Floccs Visible | 3 |
| Clumped Floccs | 4 |

Table 2 reports on the impact of core selection and surfactant treatment on flocculation.

TABLE 2

| | | Wt. % of | Appearance |
|---|---|---|---|
| Core | Surfactant | Surfactant | Ranking |
| Benzyl | — | 0 | 3 |
| Methacrylate | | | |
| Benzyl | A | 1 | 2/3 |
| Methacrylate | | | |
| Benzyl | B | 1 | 2 |
| Methacrylate | | | |
| Lauryl | — | 0 | 2 |
| Methacrylate | | | |
| Lauryl | A | 1 | 1 |
| Methacrylate | | | |
| Lauryl | B | 1 | 1 |
| Methacrylate | | | |

1. A gel composition which comprises:
(a) from 20 to 80% by weight of polyhydric alcohol,
(b) from 15 to 70% by weight of water,
(c) gelling agent,
(d) fragrance-delivery particles comprising:
i) at least one shell formed by a step-growth polymerization reaction,
ii) interior said shell, a core comprising at least one region formed by chain-grown polymerization of one or more chain growth monomers selected from the group consisting of aryl methacrylate alkyl methacrylate, and mixtures thereof, wherein the alkyl group of said alkyl methacrylate is C₆ to C₂₀ alkyl and wherein the aryl group of said aryl methacrylate is of the formula —(CH₂)ₘ—Ar where m is an integer having a value of 0 to 4 and Ar is phenyl, optionally substituted with up to three pendant alkyl groups, with the proviso that the total number of carbon atoms in the pendant alkyl groups combined does not exceed 10,
iii) optionally, surfactant grafted to said shell, wherein the fragrance delivery particles have an average diameter of less than 20 microns, and
(e) free fragrance,
wherein the composition is in the form of a deodorant stick.

2. The composition according to claim 1 wherein the shell comprises the step-growth polymerization product of one or more isocyanate monomers.

3. The composition according to claim 2 wherein the isocyanate monomers comprise isophorone diisocyanate.

4. The composition according to claim 1 wherein at least a portion of the particle shells are grafted with surfactant.

5. The composition according to claim 5 wherein the surfactant comprises non-ionic surfactant with polyglycol functionalized chains.

6. The composition according to claim 5 wherein the surfactant comprises polyethylene glycol-polypropylene glycol-polyethylene glycol block copolymer.

7. The composition according to claim 1 wherein the particles have an average diameter of less than 1 micron.

8. The composition according to claim 1 wherein the at least one region formed by chain-grown polymerization has a solubility parameter of between 17 and 19.5, inclusive.

9. The composition according to claim 1 wherein m is 0 or 1 and the alkyl group of the alkyl methacrylate is C₈ to C16 alkyl.

10. The composition according to claim 9 wherein the arylmethacrylate is unsubstituted benzyl methacrylate.

11. The composition according to claim 1 that further comprises clarifying agent.

12. A process for producing a gel composition that comprises the steps of:
a) forming an aqueous dispersion of particles comprising:
i) at least one shell formed by a step-growth polymerization reaction,
ii) interior said shell a core comprising at least one region formed by chain-grown polymerization of one or more chain growth monomers selected from the group consisting of aryl methacrylate, alkyl methacrylate, and mixtures thereof, wherein the alkyl of said methacrylate is C₆ to C₂₀ alkyland wherein the aryl group of said aryl methacrylate is of the formula —(CH₂)ₘ—Ar where is an integer having a value of 0 to 4 and Ar is phenyl, optionally substituted with up to three pendant alkyl groups, with the proviso that the total number of carbon atoms in the pendant alkyl groups combined, does not exceed 10,
iii) optionally, surfactant grafted to said shell, wherein the particles comprise at least 5% by weight of the dispersion, have an average diameter of less than 20 microns and are formed as fragrance free blanks,
b) forming a deodorant base comprising: water, polyhydric alcohol and gelling agent
c) combining the aqueous particle dispersion, deodorant base, and fragrance, fragrance optionally being added as part of the deodorant base, and
d) allowing fragrance to partition into the blanks and the resulting gel composition to solidify.

13. The composition according to claim 1 wherein the chain growth monomers are selected from the group consisting of benzyl methacrylate and lauryl methacrylate.
